(19) United States
(12) Patent Application Publication
(10) Pub. No.: US 2007/0156188 A1
(43) Pub. Date: Jul. 5, 2007

(54) DISCRIMINATION BETWEEN EFFECTIVE AND INEFFECTIVE ATRIAL PACING PULSES IN AN ACTIVE IMPLANTABLE MEDICAL DEVICE OF THE PACEMAKER TYPE

(75) Inventor: Cyrille Casset, Paris (FR)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: ELA MEDICAL S.A.S

(21) Appl. No.: 11/428,808

(22) Filed: Jul. 5, 2006

(30) Foreign Application Priority Data
Jul. 12, 2005 (FR) 05-07441

(51) Int. Cl. A61N 1/00

(52) U.S. Cl. 607/4

(57) ABSTRACT

An active implantable medical device of the cardiac pacing, resynchronization, defibrillation and/or cardio version type able to discriminate between effective and ineffective atrial pacing pulses. This device delivers atrial pacing pulses, collects an endocardial atrial signal, and senses atrial capture. It is able to recognize the presence of an evoked wave consecutive to the delivery of the pacing pulse, by an analysis of the variations of a second derivative of collected signal. A function is calculated based on integrating, over the duration of a window (F) of post-atrial pacing (StimA) atrial sensing, the absolute value of the second derivative (S″) of collected signal (S). The discrimination between effective pacing pulses and ineffective pacing pulses is performed in response to the comparison of a characteristic parameter of said function with a predetermined criterion.

### FIELD OF THE INVENTION

The present invention relates to “active implantable medical devices” as such devices are defined by the Jun. 20, 1990 Directive 90/385/CEE of the Counsel of the European Community, and more particularly to pacemakers, resynchronization, defibrillators and/or cardioverter devices intended to deliver to the heart low-energy electrical pulses for treatment of cardiac rhythm disorders.

### BACKGROUND OF THE INVENTION

Active implantable medical devices to which the present invention pertains include circuits, logic and control algorithms for detecting the activity, i.e., for detecting spontaneous depolarizations, of the myocardium, as well as means for pacing the myocardium by delivering stimulation pulses. Following such a pacing pulse, it is important to be able to collect (i.e., acquire, sense or detect) the “evoked wave”, that is, the depolarization wave induced by pacing the stimulated cavity, in order to determine whether that pacing pulse was effective. This is known as “capture” and can be used to adjust the pacing pulse energy (amplitude and/or pulse width) to ensure that the stimulation pulse will cause the stimulated cavity to contract.

In the case of atrial pacing, the search for the evoked wave is hindered due to the fact it presents an amplitude that is much lower than the evoked wave in the case of pacing the ventricle, and that in addition it is much more premature (i.e., it more closely follows in time the atrial pacing pulse relative to the delay between the ventricular stimulation and its consecutive evoked wave). One can understand that under these circumstances, it is very difficult to sense the presence of an evoked P-wave, for example, in the case of testing the atrial capture.

Some different techniques have been proposed to that end, and one can refer, for example, to European Patent EP 1433497 and its counterpart U.S. Published Patent Application US 2004/0167577 (commonly assigned herewith to ELA Medical, and incorporated herein by reference) for a description of a circuit for sensing evoked cardiac potentials consecutive to a pacing pulse. That document more specifically proposes a technique for discriminating between effective pacing pulses and ineffective pacing pulses based upon an analysis of the extrema of the second derivative of the collected atrial signal.

However, for certain configurations of the depolarization wave, the second derivative may present many significant extrema likely to interfere with the analysis and potentially might lead to a false diagnosis, positive or negative.

### OBJECTS AND SUMMARY OF THE INVENTION

It is, therefore, an object of the present invention to propose a new technique for sensing the evoked P-wave, taking into account both the very premature character and low amplitude thereof, and minimizing the risks of a false diagnosis, even in the case of atypical profiles of the evoked wave.

The device of this invention belongs to the general type described in EP 1433497 and US 2004/0167577 referred to above, that is: a device configured for delivering atrial pacing pulses, collecting an atrial endocardial signal, and sensing atrial capture and able to recognize the presence of an evoked wave consecutive to the delivery of the pacing pulse based on analysis of the variations of a second derivative of the collected signal.

In a manner characteristic of the invention, the device includes a means for sensing atrial capture which comprise means for calculation of a function integrating, over the duration of a post-atrial pacing atrial sensing window, the absolute value of the second derivative of the collected signal, and means for discriminating between effective pacing pulses and ineffective pacing pulses in response to a comparison of a parameter characteristic of said function against a predetermined criterion.

That parameter characteristic can notably be one or more of:
the final value reached by the function at the end of the post-atrial pacing atrial sensing window, and/or
the time taken for the function to reach a predetermined percentage of this final value, and/or
the slope of the function at the beginning of the post-atrial pacing atrial sensing window.

In a preferred embodiment, the device advantageously comprises means for searching the capture threshold by a dichotomy threshold search as described below.

### BRIEF DESCRIPTION OF THE DRAWINGS

Further characteristics, features and advantages of the present invention will be appreciated by a person of ordinary skill in the art in view of the following detailed discussion of a preferred embodiment of the invention, which is made with reference to the attached figures, in which the same numbers shared among all figures are referring to the same elements, and in which:

FIG. 1 is a time diagram showing the variation of a representative signal collected by the atrial sensing circuit, as well as the second derivative of that signal;

FIG. 2 is a time diagram illustrating the way the function is determined in accordance with a preferred embodiment of the present invention, this function allowing the discrimination between effective pacing pulses and ineffective pacing pulses; and

FIG. 3 is a comparative representation of the characteristic representing the evolution over time of the function of FIG. 2 for two possible cases of effective pacing pulse or ineffective pacing pulse.

### DETAILED DESCRIPTION OF THE INVENTION

Preliminarily, regarding the software-related aspects thereof, the invention can be implemented by means of an appropriate programming of the software of a known active implantable device, for example, of pacemaker or defibrillator/cardiovertor type device, comprising means for acquiring a signal provided by endocardial leads and/or implanted sensors. The invention can notably be applied to the implantable devices marketed by ELA Medical, Montrouge, France, such as the Symphony and Rhapsody brand pacemakers. These devices are equipped with programmable microprocessors, including circuits intended to acquire, format and process electrical signals collected by implanted electrodes, including an electrogram (EGM) signal, and deliver pacing pulses to these electrodes and thus to the atrial and/or ventricular cavities. It is also possible to upload towards these devices, by telemetry, software routines that will be stored in internal memory and run so as to implement the features of the invention, described in more detail below. Implementing the features of the invention into these devices is believed to be within the abilities of a person of ordinary skill in the art, and will therefore not be described in detail herein.

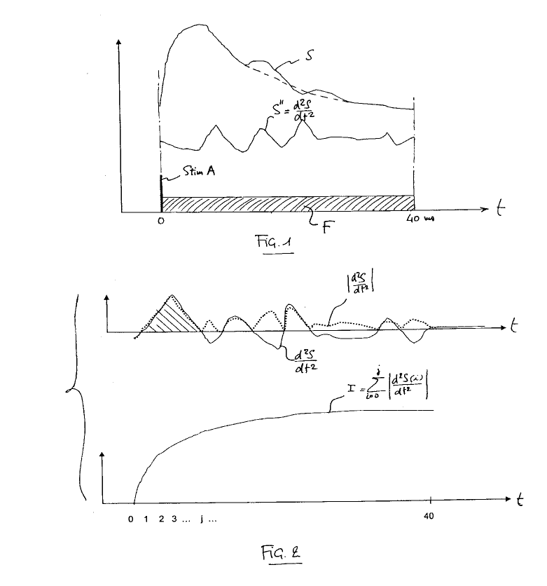

On FIG. 1, the characteristic S represents the variation of the EGM signal collected by the sensing circuits of the implanted device.

Differently from an ECG signal (surface ECG) in which the P-wave is perfectly recognizable, in the case of an EGM (endocardial), the collected signal is a complex signal, which is spread over time. This signal shows a phase of fast increase of the potential, with a duration of approximately 10 ms, followed by a phase of slow decrease spreading over approximately 30 ms. That decrease phase has a slightly uneven profile in the case when an evoked endocardial signal is actually present, as illustrated by the full line, and a less uneven profile in reverse case, as illustrated by the dashed line.

The discrimination between those two profiles will allow to determine whether or not the capture has been performed, that is to say whether the pacing pulse has been effective or not. That discrimination is done by analyzing the endocardial atrial signal S over the duration of a sensing window or “listening window” F of a fixed duration of 40 ms, for example, or parameterable (i.e. programmable) duration, e.g., in the range of from 10 to 150 ms, following an atrial pacing pulse StimA (the period referred to as “microblanking”, immediately following the delivery of the pacing pulse and during which the amplifier is switched off during a few tens of microseconds so as to avoid any load of sensing circuits, is not taken into account here).

Based upon the collected atrial endocardial signal S(t), the device determines the second derivative S″(t)=d²S/dt². That second derivative is easy to calculate based upon digitized samples, in such a way that this technique can easily and in real-time, be implemented as part of the device's microcontroller through an appropriate algorithm.

In a manner characteristic of the invention, instead of directly analyzing this second derivative S″, as described by EP 1433497 and US 2004/0167577 referred to above, which proposes to analyze the extrema of the second derivative), the device rather calculates, based upon this second derivative, a function I(t) determined as follows.

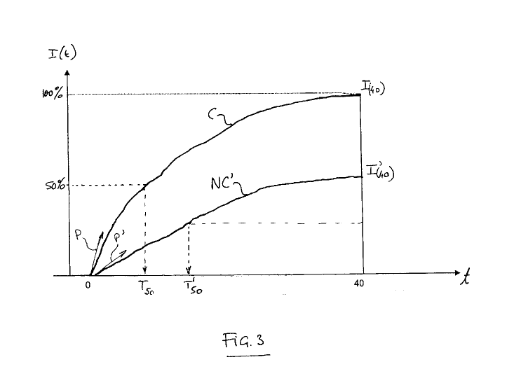

First, as illustrated by the time diagram at the top of FIG. 2, the device calculates, based upon the second derivative S″(t)=d²S/dt² (in full line), the absolute value |d²S/dt²| thereof (in dashed line).

Then, as illustrated by the time diagram at the bottom of FIG. 2, that absolute value is integrated over time during the duration of the listening window F. The signal being constituted of successive digitized samples S(1) . . . S(j) . . . S(40), that step of integration can be performed in real-time, for each successive time sample j, through a simple operation of summation of values operated by the algorithm, by calculating the following expression:

$$I ( j ) = {\sum }_{i = 0}^{j} \vert {d}^{2} S ( ⅈ ) / {dt}^{2} \vert .$$

The function I is in practice very well indicative of the effectiveness of the capture.

Thus, two typical examples of curves representing that function I are shown in FIG. 3, one (C) corresponding to a case of capture or effective, the other (NC) corresponding to a case of “no capture” or ineffective. That function I is monotonic (for it integrates a value that is always positive), and the results show that it keeps on increasing even in the end of the listening window, for the effect of a P-wave is present almost all over the duration of that window. In the case of capture, the function I is increasing faster, and reaches a higher final value.

The discrimination can be done by analyzing one or more parameters that are very representative of the obtained characteristic, notably:
the final value (respectively I(40) and I′(40)) reached at the end of the listening window, and/or
the duration (respectively T₅₀ and T′₅₀) required to reach 50% (for example) of that final value, and/or
the slope at origin (respectively P and P′), given by the initial values of the function I.

The technique described above can notably be utilized for searching a capture threshold by dichotomy. The search by dichotomy differs from the classical method of searching the threshold by delivering successive pacing pulses of decreasing energy starting from a maximum energy, until sensing the loss of capture, as described for example in European Published Patent Application EP-A-1287849 and its U.S. patent counterpart U.S. Pat. No. 6,714,820 (commonly assigned herewith to ELA Medical). This classical method has some limitations: loss of energy of the pacemaker, high number of asynchronous pacing cycles and risk of saturation of the evoked signal due to the strong energy applied through the first pacing pulses.

By operating by dichotomy, however, one will reach the searched threshold more rapidly, and the number of specific pacing pulses that are only intended to determine the capture threshold will be reduced, with a better accuracy, and therefore better information is delivered to the physician who is following the patient, when reading the data stored in the device memory. The dichotomy threshold search employs a sequence of stimulation pulse energies in which, in the absence of spontaneous atrial events, a pulse at a first energy level that is effective is followed by a pulse at a lower energy level, and a pulse at the first energy level that is ineffective is followed by a pulse at a higher energy level. This sequence continues until there are two successive decreases in stimulation in pulse energy with the second decrease stimulation pulse being ineffective.

In a preferred embodiment, the algorithm of dichotomy threshold search is as follows.

The successive atrial pacing pulses delivered with different pacing amplitudes will be referred to as A1, A2, . . . , and one will summarize the result of the capture test by Ai=OK (effective) or Ai=NOK (ineffective), corresponding to the determination of effectiveness of ineffectiveness of the applied pulse at a given amplitude, that is: whether or not it has been followed by an evoked P wave.

The first pacing pulse is applied with a minimum energy level, typically A1=0 V.

If A1=OK, then
end of the iteration (for spontaneous atrial depolarizations are present), and
increasing pacing rate until:
   A1=NOK (loss of capture), or
   authorized maximum rate reached.

If A1=NOK, then pacing pulse A2=2 V,
if A2=OK, then pacing pulse A3=1 V,
   if A3=OK, then pacing pulse A4=0,5 V,
   if A3=NOK, then A4=1,5 V and stop test.
if A2=NOK, then A3=4 V,
   if A3=OK, then A4=3 V,
   if A3=NOK, then stop test, the capture threshold is higher than 4 V.
      And the iteration pattern as above continues, until there is a detection of capture threshold.

Thus, the algorithm operates a dual dichotomy: first on the interval [0 volt, 2 volts] if A2 is effective, otherwise on the interval [0 volt, 5 volts] if A2 is ineffective. If A2 is effective, the search for a capture threshold can then be pursued over a shorter interval, and lead more rapidly to the test result.

One skilled in the art will appreciate that the present invention can be practiced by other than the described embodiments, which are presented for purposes of illustration and not of limitation.

I claim:

1. An active implantable medical device for pacing, resynchronization, defibrillation and/or cardioversion, comprising:
means for delivering atrial pacing pulses,
means for collecting an atrial endocardial signal, and
means for sensing atrial capture, able to recognize the presence of an evoked wave consecutive to a delivered pacing pulse, said sensing means comprising
means for analyzing the variations of a second derivative of the collected signal,
means for calculating a function integrating, over a duration of a window of post-atrial pacing atrial sensing, an absolute value of said second derivative, and
means for discriminating between effective pacing pulses and ineffective pacing pulses, in response to a comparison of a parameter characteristic of said function to a predetermined criterion.

2. The device of claim 1, wherein said parameter characteristic is a final value reached by said function at the end of the post-atrial pacing atrial sensing window.

3. The device of claim 1, wherein said parameter characteristic is a duration required for said function to reach a predetermined percentage of the final value reached by said function at the end of the post-atrial pacing atrial sensing window.

4. The device of claim 1, wherein said parameter characteristic is a slope of said function at the beginning of the post-atrial pacing atrial sensing window.

5. The device of claim 1, comprising further means for searching the capture threshold by a dichotomy.

6. The device of claim 5 wherein said dichotomy threshold capture searching means further comprises
means for selecting a first energy level;
means for delivering a stimulation pulse at said first energy level;
first means responsive to said first energy level stimulation pulse being effective in the absence of a spontaneous beat for selecting a lower energy for a subsequent stimulation pulse;
second means responsive to said first energy level stimulation pulse being ineffective for selecting a higher energy level for a subsequent stimulation pulse;
means for continuing to adjust the energy level using said first or second responsive means until a second successive decrease in stimulation energy results in an ineffective capture.
